(19) United States
(12) Patent Application Publication
(10) Pub. No.: US 2023/0058742 A1
(43) Pub. Date: Feb. 23, 2023

(54) METHOD AND STORAGE MEDIUM FOR QUANTITATIVE RECONSTRUCTION OF PALEOWATER DEPTH BASED ON MILANKOVITCH CYCLES

(71) Applicant: CHINA UNIVERSITY OF GEOSCIENCES, WUHAN (CN)

(72) Inventors: Qianghu LIU, Wuhan (CN); Hongtao ZHU, Wuhan (CN); Zhiyao LI, Wuhan (CN); Yuerui JIA, Wuhan (CN); Xiaobo ZHENG, Wuhan (CN); Yichen BAO, Wuhan (CN)

(21) Appl. No.: 17/857,726

(22) Filed: Jul. 5, 2022

(30) Foreign Application Priority Data
Aug. 18, 2021 (CN) 202110947608.5

(51) Int. Cl. G01N 33/24; G01N 33/18; G06F 17/11

(52) U.S. Cl. CPC G01N 33/246; G01N 33/18; G06F 17/11

(57) ABSTRACT

A method for quantitative reconstruction of paleowater depth based on Milankovitch cycles is provided, which comprises following steps: selecting a calibration well rock samples lithofacies sensitive logging data, performing major and trace element analysis, and calculating a single-point paleowater depth; denoising the lithofacies sensitive logging data; performing bandpass filter analysis and multitaper method (MTM) spectral analysis on the denoised well logging data to determine the applicability of the denoised data, followed by performing Evolutive Harmonic Analysis (Eha) & Evolutive Power Spectral Analysis and calculating Evolutive Average Spectral Misfit (eAsm) by Monte Carlo simulation, thereby obtaining a depth-domain spectrum; based on the Milankovitch astronomical cycles theory, tracking a minimum of a null hypothesis significance level; establishing an equal-depth correspondence between the obtained sedimentation rate and the single-point paleowater depth, fitting a sedimentation rate-paleowater depth equation, verifying the equation and calculating a complete sequence of paleowater depths of the calibration well.

### CROSS REFERENCE TO RELATED APPLICATION

This patent disclosure claims the benefit and priority of Chinese Patent Application No. 202110947608.5, entitled “METHOD AND STORAGE MEDIUM FOR QUANTITATIVE RECONSTRUCTION OF PALEOWATER DEPTH BASED ON MILANKOVITCH CYCLES” filed on Aug. 18, 2021, the disclosure of which is incorporated by reference herein in its entirety as part of the present application.

### TECHNICAL FIELD

The present disclosure relates to the technical field of paleowater depth reconstruction in geochemistry and geophysics, and in particular, to a method and storage medium for quantitative reconstruction of paleowater depth based on Milankovitch cycles.

### BACKGROUND ART

In a prior-art document of Yang Wanqin, Zhu Deshun, Yin Yan, and Ding Juhong, “Method for Geochemical Paleowater Depth Reconstruction and Application Thereof in Sequence Stratigraphic Division”, Geological Review, 61 (S1): 756-757, 2015, reconstruction of a paleowater depth curve is performed based on comprehensive consideration of the ratios of Ferrum (Fe)/Cobalt (Co), Fe/Manganese (Mn) and Thorium (Th)/Uranium (U) in combination with an average paleowater depth in a region. This method mainly involves steps of establishing geochemical indicators sensitive to changes in paleowater depth, integrating the geochemical indicators, performing quantitative reconstruction of paleowater depth, and utilizing the reconstructed paleowater depth curve. However, this method fails to complete water depth reconstruction of a well section with no available core data or trace element test data.

In another patent document (Application Publication No. CN106019401A) which discloses a method and a device for quantitative reconstruction of paleowater depth, the method includes the following steps: determining a beach and bar development area in a researched horizon of a paleo-lake basin; determining a single-point paleowater depth of at least one typical well in the beach and bar development area; determining a content of Th and a content of U in the at least one typical well in the beach and bar development area and calculating a ratio of Th to U from the content of Th and the content of U; performing fitting of a relational equation of the single-point paleowater depth and the ratio of Th/U based on the single-point paleowater depth of the typical well and the ratio of Th to U at the position of the single-point paleowater depth, to reconstruct quantitatively a paleowater depth of the typical well; and performing quantitative reconstruction of the paleowater depths of other wells in other areas than the beach and bar development area in the paleo-lake basin based on the relational equation of the single-point paleowater depth and the ratio of Th/U. By this method, continuous quantitative paleowater depth data of water depth changes of the paleo-lake basin in vertical and horizontal direction at the same sedimentary period can be acquired. However, limited by the particularity of the sedimentary system of beach and bar, this method fails to establish a relational equation of a paleowater depth and a ratio of Th/U for a development area without beach and bar. Besides, in this method, the selection of key parameters characterizing the paleowater depth and a reconstruction path of the paleowater depth are relatively independent.

### SUMMARY

One of major problems solved in the present disclosure is that existing methods for quantitative reconstruction of paleowater depth fail to visually reconstruct a complete sequence of water depths at the sedimentary period and thus fail to reconstruct the palaeogeomorphology and the palaeoenvironment at the sedimentary period.

According to one aspect of the present disclosure, the present disclosure provides a method for quantitative reconstruction of paleowater depth based on milankovitch cycles, including the following steps:

selecting a calibration well;

selecting a rock sample on the basis of the calibration well and acquiring lithofacies sensitive well logging data of the rock sample;

performing macro-and microelement analysis and assay on the rock sample, and calculating a single-point paleowater depth by utilizing an abundance of the macro-and microelement;

denoising the lithofacies sensitive well logging data to obtain denoised well logging data;

performing bandpass filtering analysis and multitaper method (MTM) spectral analysis in a time domain and a depth domain on the denoised well logging data, and determining whether the denoised well logging data accords with a Milankovitch astronomical signal;

if the denoised well logging data accords with the Milankovitch astronomical signal, performing Evolutive Harmonic Analysis (Eha) & Evolutive Power Spectral Analysis and calculating Evolutive Average Spectral Misfit (eAsm) by Monte Carlo simulation, thereby obtaining a depth-domain spectrum of the denoised well logging data;

based on Milankovitch astronomical cycles theory, tracking a minimum of an Asm null hypothesis significance level in eAsm program by using eAsmTrack program and obtaining a sedimentation rate in the depth-domain spectrum;

establishing an equal-depth correspondence between the obtained sedimentation rate and the single-point paleowater depth, and establishing and fitting a sedimentation rate-paleowater depth equation;

verifying the sedimentation rate-paleowater depth equation to obtain a verified sedimentation rate-paleowater depth equation; and

calculating a complete sequence of paleowater depths of the calibration well by the verified sedimentation rate-paleowater depth equation to reconstruct a spatial and temporal distribution of paleowater depths in a target area.

In some embodiments, the verifying the sedimentation rate-paleowater depth equation to obtain a verified sedimentation rate-paleowater depth equation may also include the following steps:

comparing the single-point paleowater depth calculated from the abundance of the macro-and microelement with a paleowater depth calculated by the Milankovitch's astronomical cycle equation to verify an accuracy of the sedimentation rate-paleowater depth equation.

In some embodiments, the denoising the lithofacies sensitive well logging data may also include the following steps:

in Wavelet coefficients selection 1-d program of Wavelet analysis software, decomposing the lithofacies sensitive well logging data into 9 layers by using dmey function, and removing a background a9 and a maximum frequency d1 from the layered lithofacies sensitive well logging data; and

saving the lithofacies sensitive well logging data with the background a9 and the maximum frequency d1 removed as deep-domain data containing a depth and a header and time-domain data containing no depth and no header.

In some embodiments, the performing bandpass filter analysis and MTM spectral analysis in a time domain and a depth domain on the denoised well logging data, and determining whether the denoised well logging data accords with a Milankovitch astronomical signal may also include the following steps:

performing spectral analysis on the time domain data to verify whether a characteristic peak frequency is inversely proportional to the Milankovitch cycles; and

importing the depth-domain data and using mtm code and bandpass code in astrochron software to verify whether the depth-domain data accords with the Milankovitch astronomical cycles.

In some embodiments, the depth-domain spectrum may also include information of sedimentation rates, astronomical cycles and null hypothesis significance levels.

In some embodiments, the macro-and microelement may be Co.

In some embodiments, the calculating a complete sequence of paleowater depths of the calibration well by the verified sedimentation rate-paleowater depth equation may include the following steps:

calculating complete sequences of paleowater depths of all single wells by the verified sedimentation rate-paleowater depth equation to form a distribution of paleowater depths in an entire target area extrapolated from the calibration well to all single wells; and performing quantitative reconstruction of a distribution of a paleowater depth of the entire target area at each period based on the distribution of paleowater depths in the entire target area.

In some embodiments, the establishing and fitting a sedimentation rate-paleowater depth equation may include the following steps:

integrating the single-point paleowater depth calculated from the abundance of macro-and microelement and the sedimentation rate at a depth corresponding to the paleowater depth, and fitting a functional relationship between the single-point paleowater depth and the sedimentation rate at the depth corresponding to the paleowater depth by using linear regression software like origin.

According to another aspect of the present disclosure, there is also disclosed a storage medium which is computer readable and stores thereon the method for quantitative reconstruction of paleowater depth based on Milankovitch cycles described above.

A sedimentation rate can reflect accommodation space and changes in water depth. Quantitative reconstruction of paleowater depth is realized by establishing a quantitative relationship between a paleowater depth and a sedimentation rate. Therefore, the present disclosure proposes a method for quantitatively studying paleowater depths by obtaining multi-point full-well section sedimentation rates on the basis of a sedimentation rate quantitatively calculated from Co and a relative sedimentation rate of full-well section based on the Milankovitch's theory of astronomical cycles. This method allows visual and scientific reconstruction of water depths at the sedimentary period and hence establishment of the palaeogeomorphology and the palaeoenvironment at the sedimentary period, and provides guidance for researches on sequence boundary division, sedimentation center evolution, subsidence center migration, sedimentary facies checking, etc.

### BRIEF DESCRIPTION OF THE DRAWINGS

The accompanying drawings constituting a part of the description of the present disclosure illustrate embodiments of the present disclosure, and are used together with the description to explain the principles of the present disclosure.

FIG. 1 is a flowchart of a method for quantitative reconstruction of a complete sequence of sedimentation rates and paleowater depths based on milankovitch cycles according to the present disclosure.

FIG. 2 is a flowchart of denoising well logging data according to an example of the present disclosure.

FIG. 3 is a spectrogram illustrating spectral analysis in time domain for identifying a Milankovitch astronomical signal according to an example of the present disclosure.

FIG. 4 is a spectrogram illustrating spectral analysis in depth domain for identifying a Milankovitch astronomical signal according to an example of the present disclosure.

FIG. 5 are graphs illustrating filtering analysis for identifying 405 kyr long eccentricity cycles according to an example of the present disclosure.

FIG. 6 shows an evolutive power spectrum, an evolutive harmonic spectrum, an Eha normalized amplitude spectrum according to an example of the present disclosure.

FIG. 7 shows a sedimentation rate energy spectrum and a chart illustrating a change of picked-up sedimentation rate according to an example of the present disclosure.

FIG. 8 is a diagram illustrating a functional relationship between a sedimentation rate and paleowater depth plumbing point of a verification well according to an example of the present disclosure.

FIG. 9 is a comprehensive column diagram of paleowater depths of a single well established by using a function relationship between a sedimentation rate and a paleowater depth according to an example of the present disclosure.

FIG. 10 is a distribution diagram of contour lines of paleowater depths of an interval according to an example of the present disclosure.

### DETAILED DESCRIPTION OF THE EMBODIMENTS

Exemplary embodiments of the present disclosure will be described below in detail with reference to the accompanying drawings. It should be noted that unless otherwise specified, the relative arrangement, numerical expressions and numerical values of components and steps set forth in these examples do not limit the scope of the present disclosure.

Meanwhile, it should be understood that for ease of description, various components in the accompanying drawings are not necessarily drawn to the actual scale.

The following description of at least one exemplary embodiment is merely illustrative, and not intended to limit the present disclosure and application or use thereof in any way.

To make the objectives, technical solutions, and advantages of the present disclosure clearer and more comprehensible, the present disclosure is described in further detail below with reference to specific examples and the accompanying drawings.

The technologies, methods, and equipment known to those skilled in the art may not be discussed in detail, but where appropriate, the technologies, methods, and equipment should be regarded as part of the authorized specification.

In all examples shown and discussed herein, any specific value should be interpreted as merely exemplary, rather than restrictive. Therefore, other examples of the exemplary examples may have different values.

It should be noted that similar reference numerals and letters represent similar items in the accompanying drawings below. Therefore, once an item is defined in one drawing, it does not need to be further defined and described in subsequent drawings.

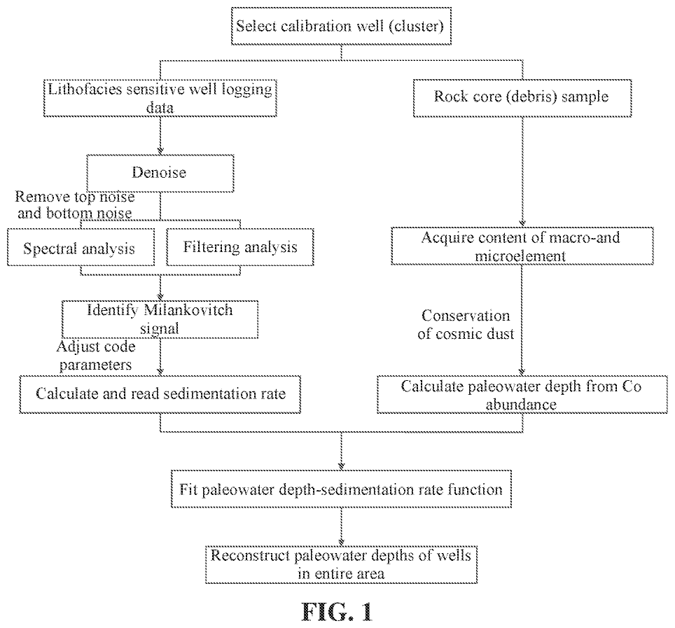

Example 1: FIG. 1 is a flowchart of a method for quantitative reconstruction of paleowater depth based on milankovitch cycles according to the example of the present disclosure. The method mainly includes the following steps.

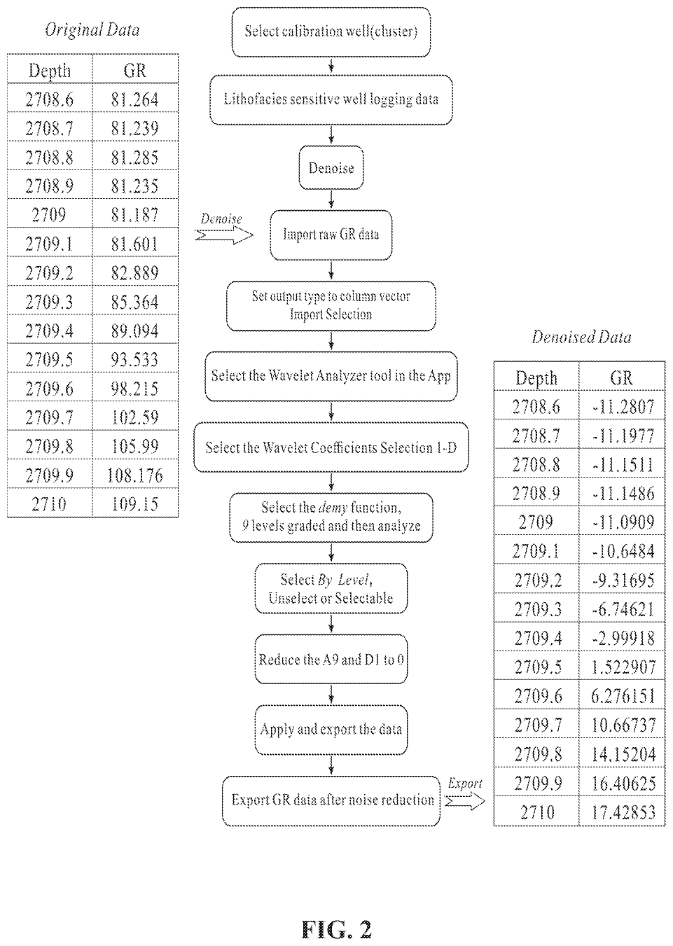

In step 1, a well with abundant data about rock core or rock debris is selected, and an appropriate lithofacies sensitivity curve, such as gamma ray (GR) curve and acoustic-velocity (AC) curve, is selected for preprocessing. FIG. 2 is a flowchart of denoising a well logging curve. After denoising, the rock core and debris are subjected to macro-and microelement analysis and assay, and a paleowater depth is calculated.

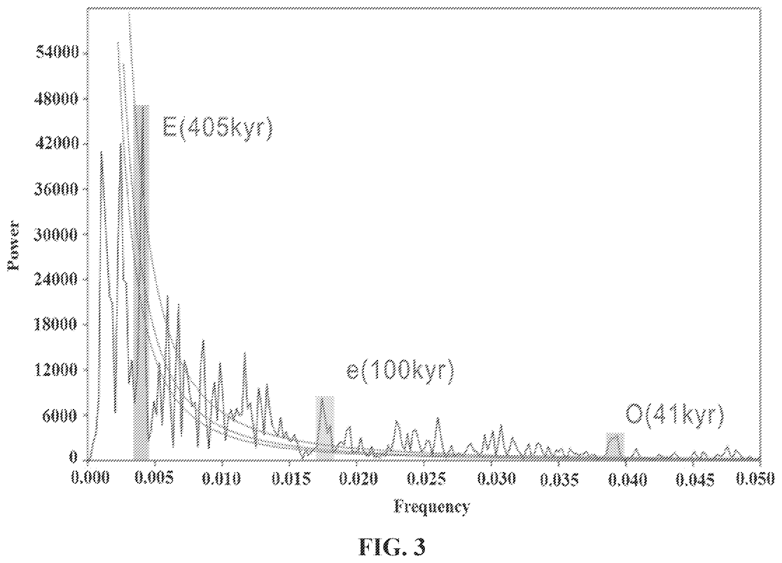

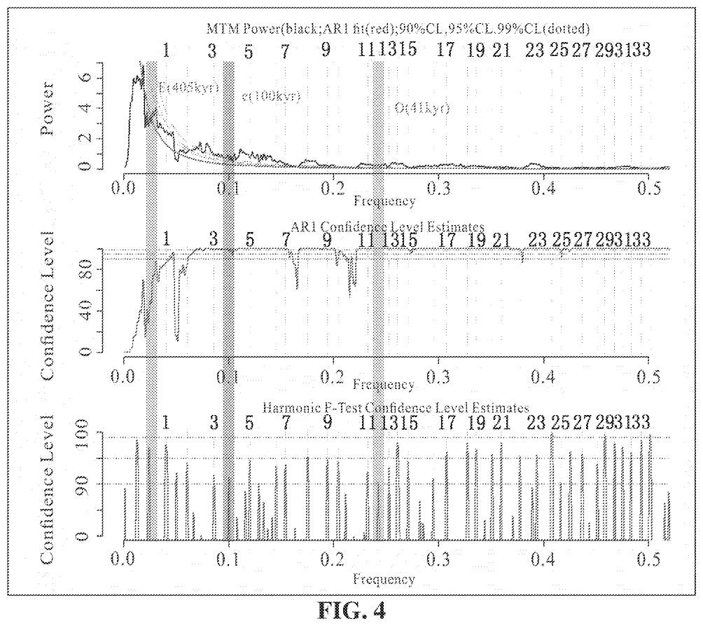

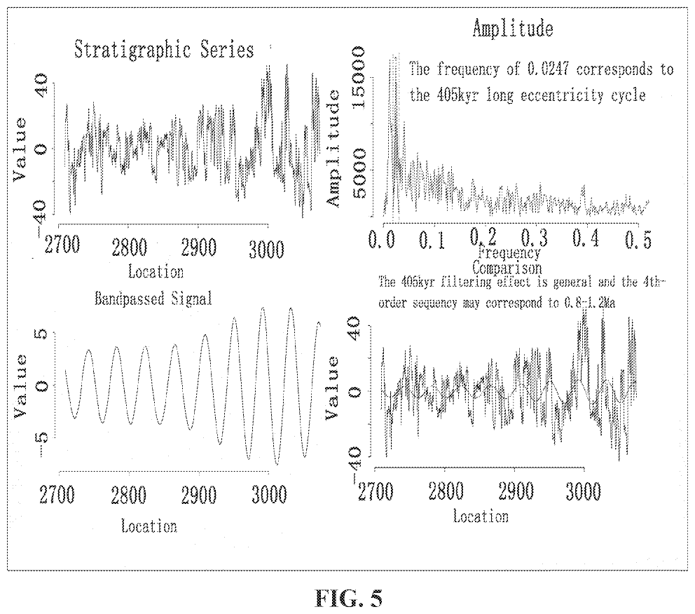

In step 2, as shown in FIGS. 3 to 5, bandpass filtering analysis and multitaper method (MTM) spectral analysis in the time domain and in the depth domain are performed on the denoised well logging data, to identify a Milankovitch astronomical signal, thereby verifying the feasibility of this method at a target interval in this area.

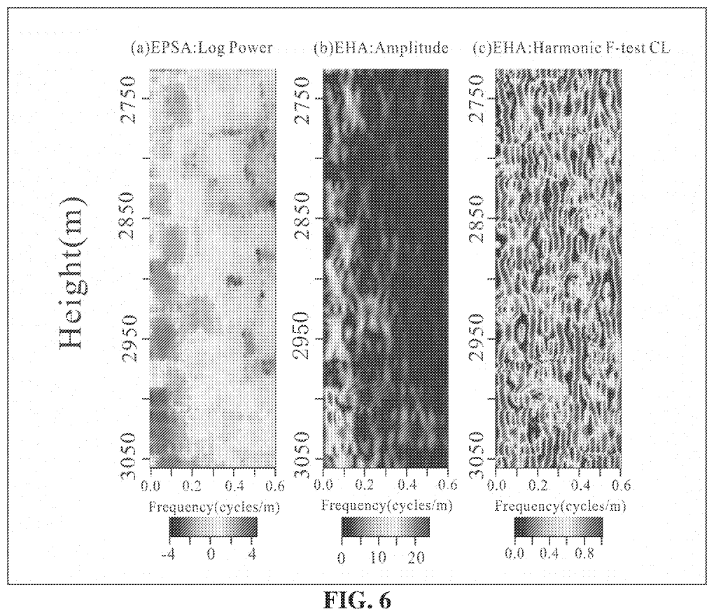

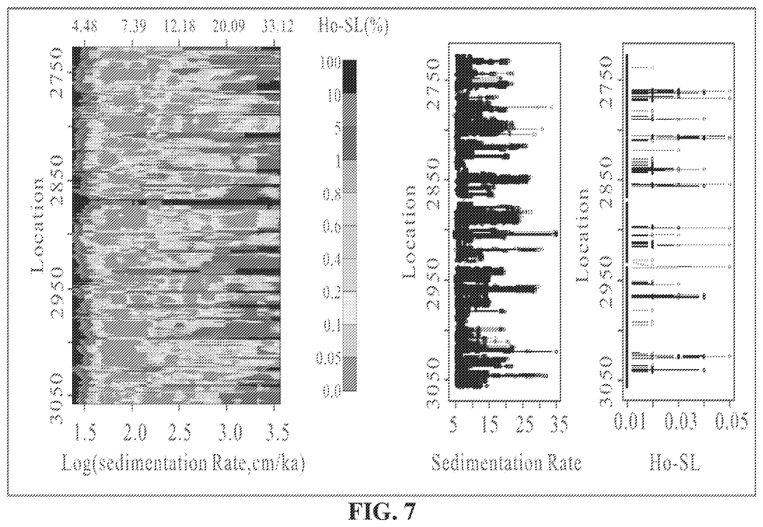

In step 3, as shown in FIG. 6, after determining that the denoised well logging data accords with the Milankovitch astronomical signal, Evolutive Average Spectral Misfit (eAsm) is calculated by Evolutive Harmonic Analysis (Eha) & Evolutive Power Spectral Analysis and monte carlo simulation to obtain the depth-domain spectrum of the denoised well logging data, the depth-domain spectrum includes the information such as sedimentation rates, astronomical cycles and null hypothesis significance levels. The minimums of the Asm null hypothesis significance levels in the eAsm program are tracked by using eAsmTrack program to obtain a sedimentation rate confidence spectrum (sedimentation rate energy spectrum) and values of sedimentation rates shown in FIG. 7.

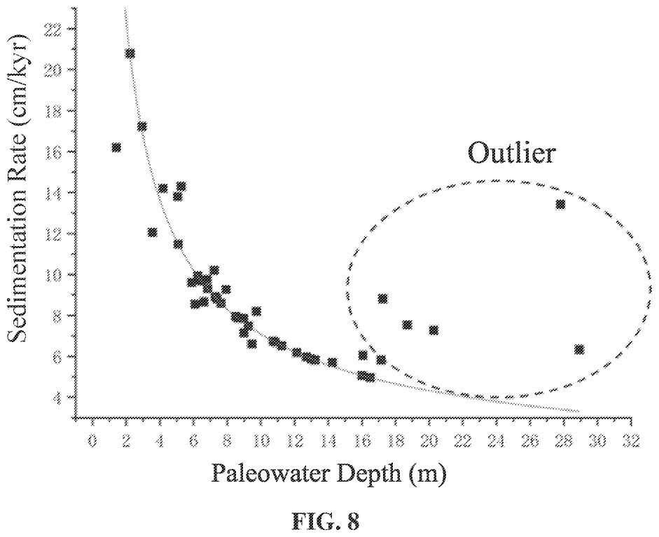

In step 4, as shown in FIG. 8, an equal-depth correspondence is established between a sedimentation rate calculated by the astronomical cycles and a paleowater depth calculated by using the abundance of Co, a function relationship between the sedimentation rate and the paleowater depth under equal-depth correspondence is fitted, and a functional relationship equation therebetween is established and fitted.

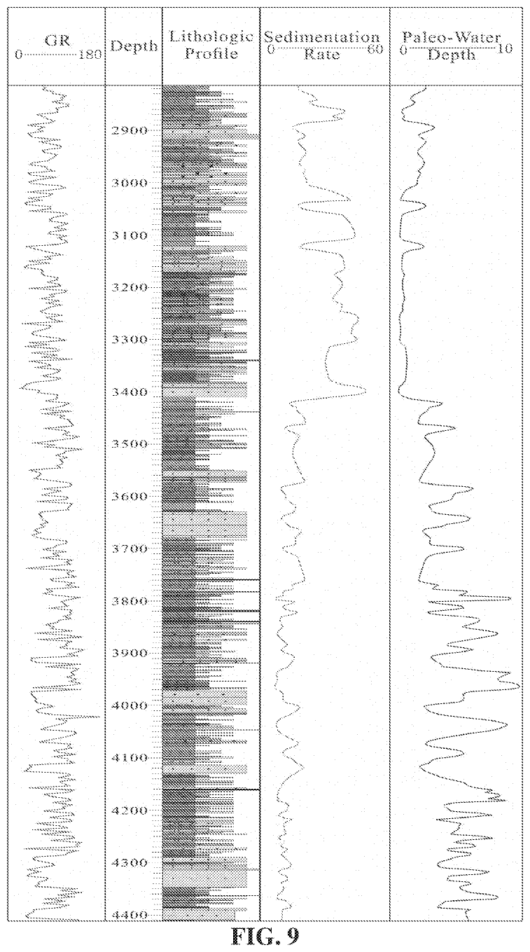

In step 5, the functional relationship equation is verified. Specifically, the paleowater depth calculated by using the abundance of Co in the rock core taken from a verification well is compared with the paleowater depth calculated by the Milankovitch's astronomical cycle equation. FIG. 9 shows a comprehensive column diagram of paleowater depths of a single well established by using the function relationship between the sedimentation rate and the paleowater depth.

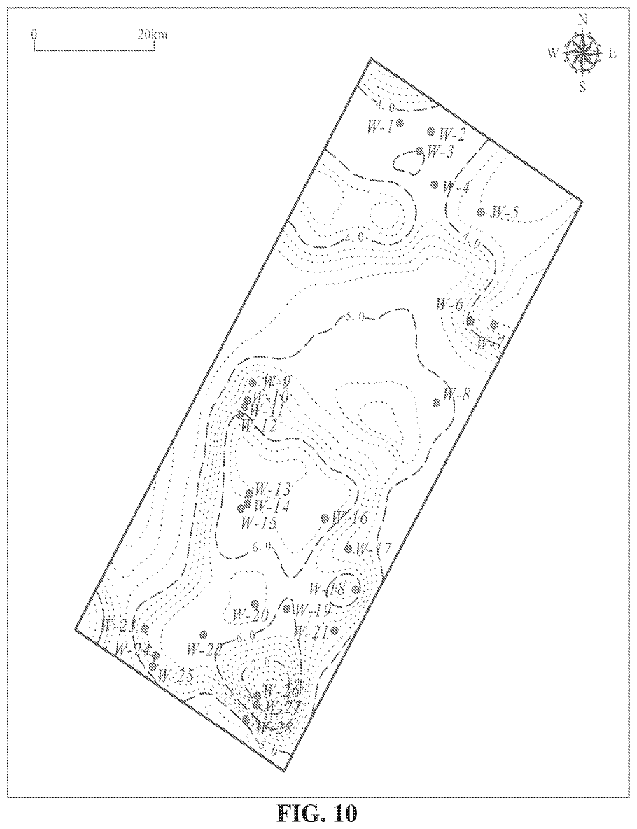

In step 6, complete sequences of paleowater depths of all wells are calculated by the function relationship equation finally obtained to realize reconstruction of spatial and temporal distribution of paleowater depths in the entire area. FIG. 10 shows the distribution diagram of contour lines of reconstructed paleowater depths of an interval.

In some examples, preferably, in the step 1 of selecting a well with abundant data about rock core or debris and selecting an appropriate lithofacies sensitivity curve (such as GR curve and AC curve) for preprocessing, the process of denoising the well logging curve, performing macro-and microelement analysis and assay on the rock core and debris and calculating the paleowater depth is specifically described as follows.

The sedimentation rate Vs of the sample is calculated by the following equation:

$${V}_{S} = \frac{{V}_{0} \times {N}_{Co}}{{S}_{Co} - t \times {T}_{Co}} \qquad ( 1 )$$

where V₀ denotes a sedimentation rate of a lake in a condition approximate to that of the modern lake, while N_{Co} denotes the abundance of Co in normal lake sediment, S_{Co} denotes the abundance of Co in the sample, T_{Co} is the abundance of Co in terrigenous clastic rock, t denotes the ratio of the content of lanthanum in the sample and the average abundance of lanthanum in the terrigenous clastic rock.

The single-point paleowater depth is calculated by the following equation:

$$h = \frac{3.05 \times {10}^{5}}{{V}_{5}^{1.5}} , \qquad ( 2 )$$

where h is the value of the single-point paleowater depth.

After the single-point paleowater depth at depth corresponding to the paleowater depth is calculated, uniformly-spaced data in resform is exported to Excel, or alternatively, data exported to Excel is subjected to uniformly spacing processing. A new table of data is created with the depth and header of the data removed, then the new table is imported to Matlab software. Denoising is performed on the data in the new table by using Wavelet coefficients selection 1-d program in Wavelet analysis software. The denoised data in the new table is decomposed into 9 layers by using dmey function, and the background a9 and the maximum frequency d1 are removed. The processed data is exported back to Excel and saved as a depth-domain version containing the depth and the header and a time-domain version containing no depth and no header.

In some examples, preferably, the step 2 of performing bandpass filtering analysis and MTM spectral analysis in the time domain and in the depth domain on the denoised data, identifying a Milankovitch astronomical signal and verifying the feasibility of this method at a target interval in this area is specifically described as follows:

The time-domain data is imported to Past3.0 (or other software capable of time-domain spectral analysis, such as Matlab and Redfit) for calculation at confidence levels of 90, 95 and 99 by using the function REDFIT in the bar of Timeseries Spectral analysis is performed to verify whether the characteristic peak frequency is inversely proportional to the Milankovitch cycle, i.e., whether the characteristic peak frequency accords with the Milankovitch's theory. The depth-domain data is then imported to R programming language (or to Rstudio software), and whether the depth-domain data accords with the Milankovitch astronomical cycles is verified by using mtm code and bandpass code in astrochron software package.

In some examples, preferably, in step 3, after determining that the data accords with the Milankovitch astronomical signal, as shown in FIG. 6, Evolutive Average Spectral Misfit (eAsm) is calculated by Evolutive Harmonic Analysis (Eha) & Evolutive Power Spectral Analysis and monte carlo simulation to obtain the depth-domain spectrum of this set of data, the depth-domain spectrum includes the information such as sedimentation rates, astronomical cycles and null hypothesis significance levels. The minimums of the Asm null hypothesis significance levels in the eAsm program are tracked by using eAsmTrack program to obtain a visual sedimentation rate spectrum, and values may be read therefrom. Specifically, Eha code, eAsm code and eAsmTrack are continuously executed on the depth-domain data in last step in the R programming language (or Rstudio software) to obtain corresponding evolutive power spectrum, evolutive harmonic spectrum, Eha normalized amplitude spectrum, sedimentation rate energy spectrum and picked-up sedimentation rate change chart, and values are preliminarily read therefrom.

In some examples, preferably, the step 4 of establishing an equal-depth correspondence between a sedimentation rate calculated from the Milankovitch astronomical cycles and a paleowater depth calculated by using the abundance of Co, and establishing and fitting a functional relationship equation therebetween is specifically described as follows: paleowater depth data is calculated by using a Co-based paleowater depth equation proposed by Wu Zhiping et al. and optimized by Zhang Caili et al. The paleowater depth data is then integrated with the sedimentation rate calculated by using the Milankovitch astronomical cycles at the corresponding depth, and the functional relationship between the paleowater depth and the sedimentation rate is fitted by using linear regression software such as origin. The paleowater depth reconstruction equation suitable for the research area of the case concerned and research areas with approximate background is as follows:

V=36.69/h^{0.714} (3)

where h is the paleowater depth calculated by using Co, while V is the sedimentation rate calculated by using the Milankovitch astronomical cycles at the corresponding depth.

The correlation coefficient R of the power function of the paleowater depth reconstruction equation is as follows: R²=0.96.

In some examples, preferably, the step 5 of verifying the equation and comparing the paleowater depth calculated from the abundance of Co in the rock core taken from a verification well with the paleowater depth calculated by the Milankovitch's astronomical cycle equation is specifically as follows:

A complete sequence of paleowater depths of the verification well is calculated by using the established sedimentation rate-paleowater depth equation, and compared with the paleowater depth calculated from the abundance of Co to verify the accuracy of the equation.

In some examples, preferably, the step 6 of calculating complete sequences of paleowater depths of all wells by the final equation to realize reconstruction of spatial and temporal distribution of paleowater depths in the entire area is specifically as follows:

The code with adjusted parameters is theoretically suitable for all wells at this interval in this area. Subsequently, the verified sedimentation rate-paleowater depth equation is utilized to calculate a complete sequence of paleowater depths of other single well to form the distribution of paleowater depths in the entire area, extrapolated across well network. Finally, the paleowater depth distribution of the entire area at each period is reconstructed.

The working principle in this example of the present disclosure is as follows: sedimentation rate can reflect changes in accommodation space and water depth. Quantitative reconstruction of paleowater depth is realized by establishing a quantitative relationship between a paleowater depth and a sedimentation rate. The single-point paleowater depth used for establishing the quantitative relationship is calculated based on Co. The sedimentation rate is calculated by using astrochron astronomical signal processing software package in the R programming language based on the Milankovitch's theory of astronomical cycles.

According to this example of the present disclosure, the paleowater depth of a well section can be calculated without coring so that the reconstruction of a complete sequence of paleowater depths of the well section with well logging curve coverage can be realized. The problem of the characteristic element method failing to calculate the paleowater depths of the well section without coring is solved. Paleowater depths are quantitatively calculated from information about the well, such that the sedimentation environments and evolution of different tectonic-sedimentary units at different geological periods can be characterized visually and scientifically, to indicate changes in accommodation space and simulate the process of sedimentation, thereby establishing an evolutionary series of sedimentation water depths. Besides, this method can be used in guidance and verification for the reconstruction of palaeogeomorphology to a certain extent.

A change in paleowater depth directly reflects a change in lake (sea) level and is closely associated with cyclostratigraphy, and its research precision can be at the level of dozens of thousands of years (ky), so as to provide a new idea for quantitative reconstruction of the high-frequency sequence stratigraphic framework of each tectonic unit. After each tectonic unit is processed by using this method, a sedimentation rate-paleowater depth change curve may also be made for a well section to visually and clearly display changes in lake (sea) level, thereby guiding a prediction of advantageous sand distribution and a sequencing of preferred targets. Furthermore, a visual method with strong operability is provided for division of sequence stratigraphic frameworks and prediction of favorable source rocks and reservoir bodies with seismic data and geophysical data. The problem of insufficient microelement samples involved in oil-gas exploration is solved. In short, this method can be extensively used and has broad market prospects.

The combination of geophysical data with the Milankovitch's theory of astronomical cycles is the latest achievement of geologists and palaeoclimatologists in the present century and the new idea in the field of this discipline. This method has the advantages of good operability, strong pertinence, etc. The present disclosure provides a spectrum generating flow with strong operability for reconstruction of paleowater depths of a sedimentary area with well logging data, describes the working concepts, principles, methods and flow in detail, and reflects new ideas and new methods of utilizing the Milankovitch's theory in palaeoenvironment reconstruction. This method has the characteristics of advancement, exploration utility, and wide applicability and can be widely applied in studies of sequence stratigraphy and sedimentology and in petroleum geology exploration.

The foregoing is merely illustrative of the preferred examples of the present disclosure and is not intended to limit the present disclosure. Any modification, equivalent replacement and improvement made without departing from the spirit and principle of the present disclosure shall be included within the protection scope of the present disclosure.

It should also be noted that the term “comprise”, “include”, or any other variant thereof is intended to encompass a non-exclusive inclusion, such that a process, method, product, or device that includes a series of elements includes not only those elements, but also other elements not explicitly listed, or elements that are inherent to such a process, method, product, or device. Without more restrictions, an element defined by the phrase “including a . . . ” does not exclude the presence of another same element in a process, method, product, or device that includes the element.

1. A method for quantitative reconstruction of paleowater depth based on Milankovitch cycles, comprising:
selecting a calibration well;
selecting a rock sample based on the calibration well and acquiring lithofacies sensitive well logging data of the rock sample;
performing macro-and microelement analysis and assay on the rock sample, and calculating a single-point paleowater depth by utilizing an abundance of the macro-and microelement;
denoising the lithofacies sensitive well logging data to obtain denoised well logging data;
performing bandpass filtering analysis and multitaper method (MTM) spectral analysis in a time domain and a depth domain on the denoised well logging data, and determining whether the denoised well logging data accords with a Milankovitch astronomical signal;
if the denoised well logging data accords with the Milankovitch astronomical signal, performing Evolutive Harmonic Analysis (Eha) & Evolutive Power Spectral Analysis and calculating Evolutive Average Spectral Misfit (eAsm) by Monte Carlo simulation, thereby obtaining a depth-domain spectrum of the denoised well logging data;
based on Milankovitch astronomical cycle theory, tracking a minimum of a null hypothesis significance level and obtaining a sedimentation rate in the depth-domain spectrum;
establishing an equal-depth correspondence between the obtained sedimentation rate and the single-point paleowater depth, and establishing and fitting a sedimentation rate-paleowater depth equation;
verifying the sedimentation rate-paleowater depth equation to obtain a verified sedimentation rate-paleowater depth equation; and
calculating a complete sequence of paleowater depths of the calibration well by the verified sedimentation rate-paleowater depth equation to reconstruct a spatial and temporal distribution of paleowater depths in a target area.

2. The method for quantitative reconstruction of paleowater depth based on Milankovitch cycles according to claim 1, wherein the verifying the sedimentation rate-paleowater depth equation to obtain a verified sedimentation rate-paleowater depth equation comprises:
comparing the single-point paleowater depth calculated from the abundance of the macro-and microelement with a paleowater depth calculated by the Milankovitch's astronomical cycle equation to verify an accuracy of the sedimentation rate-paleowater depth equation.

3. The method for quantitative reconstruction of paleowater depth based on Milankovitch cycles according to claim 1, wherein the denoising the lithofacies sensitive well logging data comprises:
decomposing the lithofacies sensitive well logging data into 9 layers by using dmey function, and removing a background a9 and a maximum frequency d1 from the layered lithofacies sensitive well logging data; and
saving the lithofacies sensitive well logging data with the background a9 and the maximum frequency d1 removed as deep-domain data containing a depth and a header and time-domain data containing no depth and no header.

4. The method for quantitative reconstruction of paleowater depth based on Milankovitch cycles according to claim 3, wherein the performing bandpass filtering analysis and MTM spectral analysis in a time domain and a depth domain on the denoised well logging data, and determining whether the denoised well logging data accords with a Milankovitch astronomical signal comprise:
importing the time-domain data for spectral analysis to verify whether a characteristic peak frequency is inversely proportional to the Milankovitch cycles; and
importing the depth-domain data and using mtm code and bandpass code in astrochron software to verify whether the depth-domain data accords with the Milankovitch astronomical cycles.

5. The method for quantitative reconstruction of paleowater depth based on Milankovitch cycles according to claim 1, wherein the depth-domain spectrum comprises information of sedimentation rates, astronomical cycles and null hypothesis significance levels.

6. The method for quantitative reconstruction of paleowater depth based on Milankovitch cycles according to claim 1, wherein the macro-and microelement is Co.

7. The method for quantitative reconstruction of paleowater depth based on Milankovitch cycles according to claim 1, wherein the calculating a complete sequence of paleowater depths of the calibration well by the verified sedimentation rate-paleowater depth equation comprises:
calculating complete sequences of paleowater depths of all calibration wells by the verified sedimentation rate-paleowater depth equation to form a distribution of paleowater depths in an entire target area extrapolated from the calibration well to all calibration wells; and
performing quantitative reconstruction of a distribution of a paleowater depth of the entire target area at each period based on the distribution of paleowater depths in the entire target area.

8. The method for quantitative reconstruction of paleowater depth based on Milankovitch cycles according to claim 1, wherein the establishing and fitting a sedimentation rate-paleowater depth equation comprises:
integrating the single-point paleowater depth calculated from the abundance of the macro-and microelement and the sedimentation rate at a depth corresponding to the paleowater depth, and fitting a functional relationship between the single-point paleowater depth and the sedimentation rate at the depth corresponding to the paleowater depth by using linear regression software.

9. A storage medium which is computer readable and stores thereon a method for quantitative reconstruction of paleowater depth based on Milankovitch cycles, wherein the method comprises:
selecting a calibration well;
selecting a rock sample based on the calibration well and acquiring lithofacies sensitive well logging data of the rock sample;
performing macro-and microelement analysis and assay on the rock sample, and calculating a single-point paleowater depth by utilizing an abundance of the macro-and microelement;
denoising the lithofacies sensitive well logging data to obtain denoised well logging data;
performing bandpass filtering analysis and multitaper method (MTM) spectral analysis in a time domain and a depth domain on the denoised well logging data, and determining whether the denoised well logging data accords with a Milankovitch astronomical signal;
if the denoised well logging data accords with the Milankovitch astronomical signal, performing Evolutive Harmonic Analysis (Eha) & Evolutive Power Spectral Analysis and calculating Evolutive Average Spectral Misfit (eAsm) by Monte Carlo simulation, thereby obtaining a depth-domain spectrum of the denoised well logging data;
based on Milankovitch astronomical cycle theory, tracking a minimum of a null hypothesis significance level and obtaining a sedimentation rate in the depth-domain spectrum;
establishing an equal-depth correspondence between the obtained sedimentation rate and the single-point paleowater depth, and establishing and fitting a sedimentation rate-paleowater depth equation;
verifying the sedimentation rate-paleowater depth equation to obtain a verified sedimentation rate-paleowater depth equation; and
calculating a complete sequence of paleowater depths of the calibration well by the verified sedimentation rate-paleowater depth equation to reconstruct a spatial and temporal distribution of paleowater depths in a target area.

10. The storage medium according to claim 9, wherein the verifying the sedimentation rate-paleowater depth equation to obtain a verified sedimentation rate-paleowater depth equation comprises:
comparing the single-point paleowater depth calculated from the abundance of the macro-and microelement with a paleowater depth calculated by the Milankovitch's astronomical cycle equation to verify an accuracy of the sedimentation rate-paleowater depth equation.

11. The storage medium according to claim 9, wherein the denoising the lithofacies sensitive well logging data comprises:
decomposing the lithofacies sensitive well logging data into 9 layers by using dmey function, and removing a background a9 and a maximum frequency d1 from the layered lithofacies sensitive well logging data; and
saving the lithofacies sensitive well logging data with the background a9 and the maximum frequency d1 removed as deep-domain data containing a depth and a header and time-domain data containing no depth and no header.

12. The storage medium according to claim 11, wherein the performing bandpass filtering analysis and MTM spectral analysis in a time domain and a depth domain on the denoised well logging data, and determining whether the denoised well logging data accords with a Milankovitch astronomical signal comprise:
importing the time-domain data for spectral analysis to verify whether a characteristic peak frequency is inversely proportional to the Milankovitch cycles; and
importing the depth-domain data and using mtm code and bandpass code in astrochron software to verify whether the depth-domain data accords with the Milankovitch astronomical cycles.

13. The storage medium according to claim 9, wherein the depth-domain spectrum comprises information of sedimentation rates, astronomical cycles and null hypothesis significance levels.

14. The storage medium according to claim 9, wherein the macro-and microelement is Co.

15. The storage medium according to claim 9, wherein the calculating a complete sequence of paleowater depths of the calibration well by the verified sedimentation rate-paleowater depth equation comprises:
calculating complete sequences of paleowater depths of all calibration wells by the verified sedimentation rate-paleowater depth equation to form a distribution of paleowater depths in an entire target area extrapolated from the calibration well to all calibration wells; and
performing quantitative reconstruction of a distribution of a paleowater depth of the entire target area at each period based on the distribution of paleowater depths in the entire target area.

16. The storage medium according to claim 9, wherein the establishing and fitting a sedimentation rate-paleowater depth equation comprises:
integrating the single-point paleowater depth calculated from the abundance of the macro-and microelement and the sedimentation rate at a depth corresponding to the paleowater depth, and fitting a functional relationship between the single-point paleowater depth and the sedimentation rate at the depth corresponding to the paleowater depth by using linear regression software.
